(19) United States
(12) Patent Application Publication
(10) Pub. No.: US 2011/0243788 A1
(43) Pub. Date: Oct. 6, 2011

(54) FRESH SCENTS AIR FILTERS SYSTEMS

(76) Inventor: Jerry Garten, Medicine Lodge, KS (US)

(21) Appl. No.: 13/079,659

(22) Filed: Apr. 4, 2011

Related U.S. Application Data
(60) Provisional application No. 61/320,843, filed on Apr. 5, 2010.

(51) Int. Cl. A61L 9/00

(52) U.S. Cl. 422/4; 422/123

(57) ABSTRACT

A scented air filters system comprising a cardboard frame wherein a porous filter media is housed for use in filtering airborne particulates from the air where ever air conditioning systems, furnaces, and evaporative cooling systems may be utilized. The filters are for use in both residential and commercial buildings. A breakably-actuated scent container is located in the squeezably-deformable cardboard frame having porous filter media. The scent containers are activated when fractured by squeezing the frame between the thumb and fingers before installing the filter in the vent housing. The scented material is scented liquid comprising oil, non-oil, granulated solids, or gel(s). Fragrance is dispersed as air flows through the filter and ducting system.

### CROSS-REFERENCE TO RELATED APPLICATION

The present application is related to and claims priority from prior provisional application Ser. No. 61/320,843, filed Apr. 5, 2010 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of air filters and more specifically relates to scented air filters that are used with air flow systems.

### 2. DESCRIPTION OF THE RELATED ART

Modern residential and commercial buildings use air handling systems to move air within them. Occupants often look for ways to freshen these areas. Many things in modern day homes create or tend to create odors that may be unpleasant and potentially dangerous to human health. Our modern conveniences provide a quality of life for the average member of society that was heretofore unknown before but by the very nature of some of the conveniences, require constant maintenance. For example, garbage baskets often tend to start smelling before they are taken out. Moisture in carpets in the warmer climates may produce unpleasant odors that dominate the interior of a home. Bathrooms with inadequate ventilation tend to become a problem for the whole house. The owner of a home, no matter how conscious of unpleasant odors, will typically become desensitized to the odors within his or her own home. This may lead to health problems and embarrassing situations. A modern practice is to use various types of air fresheners to combat the problem; however there are drawbacks to this proposed solution.

Air fresheners are consumer products that may be used in homes or commercial building rooms that typically emit fragrance. There are two broad air freshener categories: continuous action and instant action. Continuous action products may include: scented candles and devices which use a candle flame or some other heat source to heat and vaporize a fragrance formulation. Continuous action products may also include incense burners; wall plug-ins which either use Piezoelectricity technology to aerosolize fragrance or heat to vaporize it; fragrance impregnated gels which release fragrance as the gel evaporates (sometimes with the help of an electric fan); wick and reed diffusers which release fragrance by evaporation from fragrance-soaked wicks or wooden reeds. Other options of this form of product may comprise fragrance impregnated materials like floor wax, paper, plastics, and wood which release fragrance by off-gassing; and lastly nebulization systems which convert liquid fragrances to a vapor in a cold process without the use of heat.

Typically, instant action systems are mainly aerosol sprays, or atomizers. The aerosol spray normally uses a propellant and fragrance packaged under pressure. These sprays may be contained within a sealed metal or glass container with a valve which is opened by pressing down a button which contains a spray nozzle or actuator. When the container's valve is opened by pressing the actuator, fragrance is forced through the spray nozzle located inside the actuator to create a mist of droplets containing fragrance. These droplets are typically 30 to 50 micrometres in diameter.

The atomizer may be a glass, metal or plastic container of fragrance which operates in a similar fashion except that the actuator is a pump which when pressed a few times creates the pressure to aspirate the fragrance from the container through a tube into the actuator and spray nozzle. The mist created may also contain droplets 50 to 100 micrometres in diameter. Many individuals purchase sprays that only temporarily scent a specific room. Moving from room to room to spray these devices can be time consuming and inconvenient. Purchasing replacement canisters can become costly and fill landfills prematurely. Using scented candles can be dangerous if the candle is left burning unattended, especially in a house with children and pets. Homeowners that have central air are supposed to replace their air filters on a regular basis. Many forget to do so, which can be harmful to both the system and the individual. A solution to these problems is desirable.

Various attempts have been made to solve the above-mentioned problems such as those found in U.S. Pat. Nos. 4,875,912; 4,604,114; 4,563,333; 4,065,262; 4,118,226; and 5,258,051. This prior art is representative of scent emitting air filters. None of the above inventions and patents, taken either singly or in combination, is seen to describe the invention as claimed.

Ideally, an air filter should be multiple functional and yet, would operate reliably and be manufactured at a modest expense. Thus, a need exists for a reliable scented air filter system to filter airborne particulates and provide a pleasant scent, and to avoid the above-mentioned problems.

### BRIEF SUMMARY OF THE INVENTION

In view of the foregoing disadvantages inherent in the known air filter art, the present invention provides a novel scented air filter system. The general purpose of the present invention, which will be described subsequently in greater detail is to provide convenience and reduced costs for creating aromatic scents throughout the interior of a building.

The present scented air filter system as described herein preferably comprises a frame (preferably comprised of cardboard) for housing a porous filter media and at least two breakably-actuated scent containers affixed within the frame. The breakably-actuated scent containers are preferably fracturable tubes containing a liquid scent, but in other embodiments may be a non-liquid (gasses or solids.) The frame may comprise two vertically parallel channels and two horizontally parallel channels in which two of the breakably-actuated scent containers are locatable within the confines of the two vertically and/or two horizontally parallel channels. The frame is squeezably-deformable (cardboard embodiments) such that when the breakably-actuated scent containers are squeezed within the frame, they release a liquid from the tube(s) wherein the liquid wicks into an absorbent material exposed to the flow of air within the ducting.

Further, the scented air filter system may be usable as a particulate impeder for a cold air suction on a furnace, for a wall vent, and/or for a cold air suction on an air conditioner. The breakably-actuated scent container comprises a liquid that readily flows. The porous filter media comprises a wicking volume, defined by a first and a second outer surface. The flowable liquid from the breakably-actuated scent container is moved by capillary action from the wicking volume (when absorbed) towards the first and the second outer surfaces whereby the liquid is dispersible by airflow moving through the ducting. The liquid within the breakably-actuated scent container in different embodiments may comprise oil or a non-oil fluid, or a granular material. Alternate embodiments may include a scented gel. The usable duration of the breakably-actuated scent container is measured at a point from the breakable-actuation to a no-scent-release condition which coincides with one filter change-out interval and provides an indication of a necessary maintenance task. The frame houses and defines an outer perimeter for the porous filter media wherein the breakably-actuated scent container is located within inner confines of the frame. When the breakably-actuated scent container is breakably-actuated, a dispersible fragrance is created and moved by an air flow throughout the interior building spaces.

A kit is also described the kit having scented air filters of varying scents/forms for use with furnaces and vents; and a set of user instructions.

A method of using a scented air filter system is also disclosed herein comprising the steps of: removing a used filter from a filter housing when no scent is being released; squeezing or otherwise deforming the sides of a frame (of a new filter) to fracture at least one breakably-actuated scent container to allow scenting material to emit a scent; inserting said new filter into said filter housing; and disposing of used filter.

The present invention holds significant improvements and serves as a scented air filter(ing) system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, Fresh Scents Air Filters Systems, constructed and operative according to the teachings of the present invention.

FIG. 1 is a perspective view illustrating a Fresh Scents Air Filters System(s) in an in-use condition using a flow diagram showing how freshening scents are distributed throughout an interior of a building according to an embodiment of the present invention.

FIG. 2 is a perspective view illustrating various sized framed filters of the Fresh Scents Air Filters Systems according to an embodiment of the present invention of FIG. 1.

FIG. 3 is a perspective view illustrating cardboard framing and relative location of a breakably-actuated scent container within the frame of the Fresh Scents Air Filters Systems according to an embodiment of the present invention of FIG. 1.

FIG. 4 is a perspective view illustrating the scent container being breakably-actuated according to an embodiment of the present invention of FIGS. 1-3.

FIG. 5 is a flowchart illustrating a method of use according to an embodiment of the present invention of FIG. 1.

The various embodiments of the present invention will hereinafter be described in conjunction with the appended drawings, wherein like designations denote like elements.

### DETAILED DESCRIPTION

As discussed above, embodiments of the present invention relate to an air filtering system and more particularly to a scented air filter system as used to improve the maintenance of air filters and provide convenience of using air fresheners.

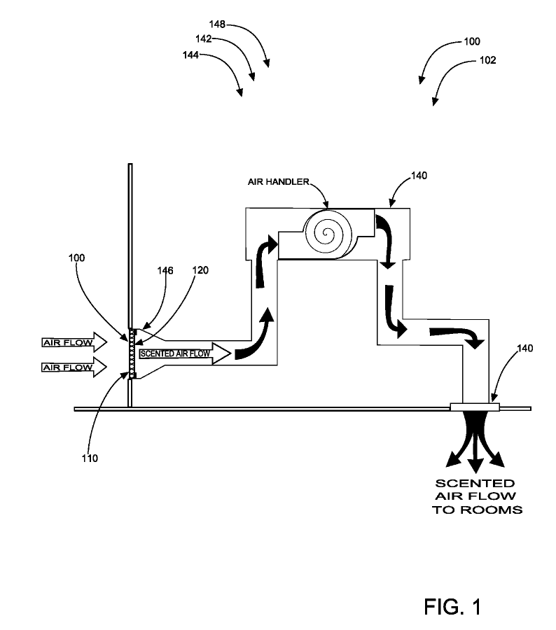

Referring now to FIG. 1, a perspective view illustrating Fresh Scents Air Filters System(s) 100 in an in-use condition using a flow diagram showing how freshening scents may be distributed throughout an interior of a building (via the existing air ducts 140 of furnace 142 or air conditioning system 144) according to an embodiment of the present invention. Fresh scents air filters systems 100 preferably comprises: frame 110; porous filter media 120; and breakably-actuated scent container 130; wherein frame 110 houses and defines an outer perimeter of porous filter media 120. Breakably-actuated scent container 130 is preferably located within frame 110 such that when breakably-actuated, creates a dispersible fragrance moveable by an air flow. A usable duration of breakably-actuated scent container 130 upon breakable-actuation approximately equals one Filter(s) 114 change-out interval, thereby creating an indication to a building owner that the filter needs to be changed.

Filter(s) 114, which are preferably scent-infused also serve as a pleasant reminder for individuals to replace their filter(s) 114. When the scent can no longer be detected, it is time to change the filter(s) 114. Because the scent will dissipate over time, individuals may be thereby alerted when a replacement of filter(s) 114 is needed. This innovative item provides a simple and effective way to revitalize the circulated air in buildings with an existing heating and cooling system. Fresh scents air filters systems 100 is also usable as a particulate impeder for a cold air suction on furnace 142, on wall vent 146, or a cold air suction on an air conditioning system 144. The general flow of air through a building's air circulation system is shown as used with the present invention.

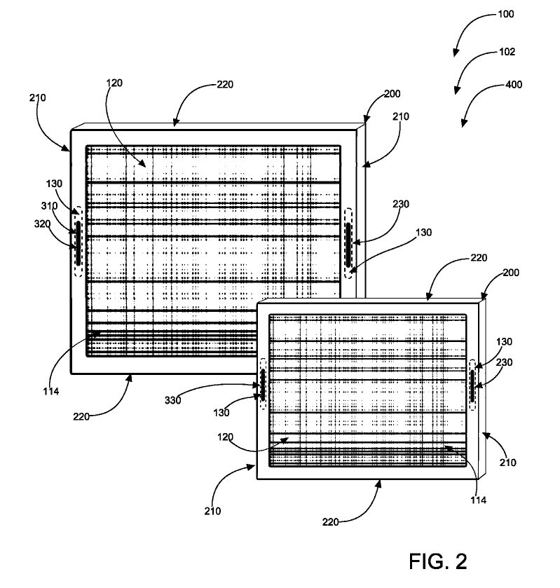

Referring now to FIG. 2, a perspective view illustrating various sizes of filter(s) 114 available with fresh scents air filters systems 100 according to an embodiment of the present invention of FIG. 1.

Filter(s) 114 may be available in various standard filter(s) 114 sizes for furnace(s) 142 and/or air conditioning systems 144 sized to fit most manufactured brands, as alluded to in the present figure. Frame 110 of filter(s) 114 may be comprised of cardboard 200 and preferably has two vertically parallel channels 210 and two horizontally parallel channels 220 having at least two breakably-actuated scent containers 130 located within the two vertically parallel channels 210. Other embodiments may have two of breakably-actuated scent containers 130 located within two horizontally parallel channels 220 or may have four of the breakably-actuated scent containers 130 located within all four sides of filter(s) 114 in preferred embodiments. Porous filter media 120 comprises a wicking volume defined by a first and a second outer surface; wherein liquid 230 from breakably-actuated scent container 130 is moved by capillary action from the wicking volume towards the first and second outer surfaces whereby liquid 230 is dispersible by airflow moving through air ducts 140 system. In this manner the scent/fragrance may be introduced to the air flow and thereby circulated.

Referring also to FIG. 2, showing fresh scents air filters systems 100 which may be sold as kit 400 comprising the following parts: at least one set of air filters 114 (or varying sizes, scent-packet locations and other) and at least one set of user instructions. Accordingly, fresh scents air filters systems 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

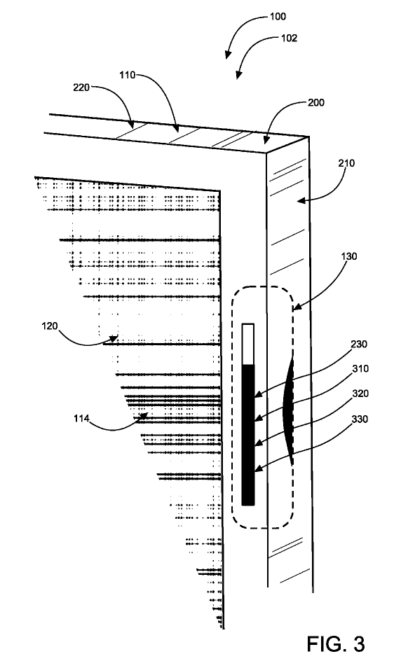

Referring now to FIG. 3, a perspective view illustrating frame 110 preferably made of cardboard 200 and relative location of breakably-actuated scent container 130 within frame 110 according to an embodiment of the present invention of FIG. 1.

Within the present invention a wide variety of scents may be available, including spices, flowers and others. Users may break breakably-actuated scent containers 130 before inserting filter(s) 114 in place. Breakably-actuated scent container 130 may be comprised of at least one tube containing oil 310 that maintains a scent for extended durations of time. In other embodiments breakably-actuated scent container 130 may contain non-oil liquid 320 or granular material 330 that slowly dissolves over time and disperses its scent throughout the air-flow stream. The scent may dissipate after a week or a month, depending on the HVAC system, air conditioning system 144, or evaporative cooling system 148. Breakably-actuated scent containers 130 may be added to all four sides of frame 110 on larger filter(s) 114 or on commercially available filter(s) 114.

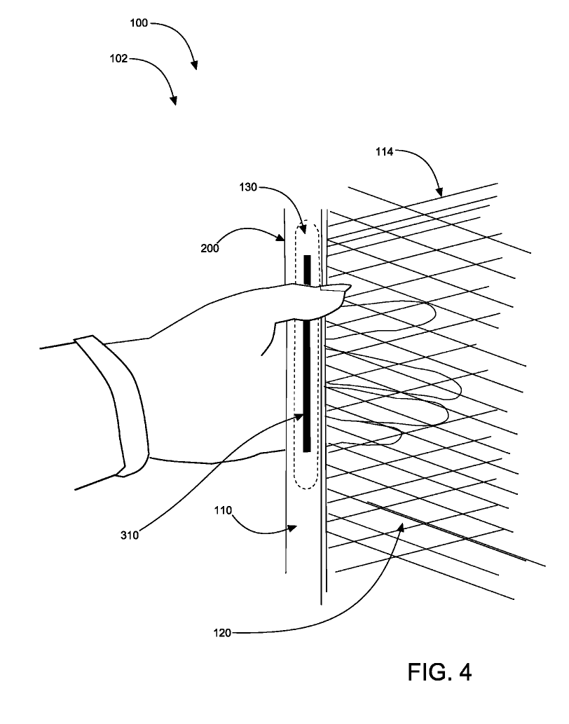

Referring now to FIG. 4, a perspective view illustrating breakably-actuated scent containers 130 of fresh scents air filters systems 100 according to an embodiment of the present invention of FIG. 1.

The invention preferably comprises an (air) filter(s) 114 preferably with integrated breakably-actuated scent containers 130 in its (cardboard 200) frame 110. Frame 110 may be deformably squeezed or otherwise manipulated between the thumb and fingers in order to break or fracture the brittle breakably-actuated scent containers 130, which will cause the scent oil 310 to wick outwardly into porous filter media 120, penetrating porous filter media 120, and exposing it to the air flow stream dispersing the scent. Breakably-actuated scent container 130 may be embedded within cardboard 200 frame 110 in vertically parallel channels 210 or may be proximate to the center of porous filter media 120 and closer to the center of air-flow in other embodiments. Frame 110 is squeezably-deformable such that when breakably-actuated scent container is squeezed within frame 110, breakably-actuated scent container 130 is breakably-actuated to release liquid 230.

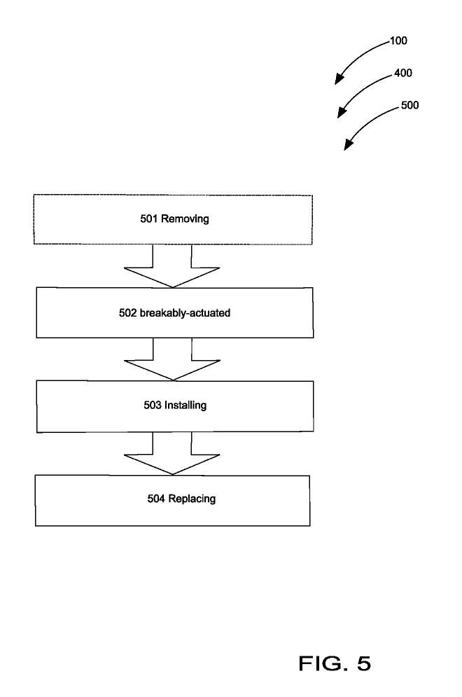

Referring now to FIG. 5, a flowchart illustrating a method of use 500 for fresh scents air filters systems 100 comprising the steps of: step one 501 removing a depleted air filter(s) 114 from a filter vent housing; step two 502 breakably-actuating breakably-actuated scent containers 130 on (new) filter(s) 114; step three 503 installing (new) filter(s) 114; and step four 504 replacing filter(s) 114 vent cover. It should be noted that step 501 is an optional step and may not be implemented in all cases. Optional steps of method 500 are illustrated using dotted lines in FIG. 5 so as to distinguish them from the other steps of method 500.

It should be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A scented air filter system comprising:
a frame;
a porous filter media; and
a breakably-actuated scent container;
wherein said frame houses and defines an outer perimeter of said porous filter media;
wherein said breakably-actuated scent container is located within said frame; and
wherein said breakably-actuated scent container when breakably-actuated creates a dispersible fragrance moveable by an air flow.

2. The scented air filter system of claim 1 wherein said breakably-actuated scent container is embedded within said porous filter media.

3. The scented air filter system of claim 1 wherein said breakably-actuated scent container comprises a liquid.

4. The scented air filter system of claim 1 wherein said breakably-actuated scent container comprises a granular material.

5. The scented air filter system of claim 3 wherein said liquid within said breakably-actuated scent container comprises an oil.

6. The scented air filter system of claim 3 wherein said frame is squeezably-deformable such that when said breakably-actuated scent container is squeezed within said frame, said breakably-actuated scent container is breakably-actuated to release said liquid.

7. The scented air filter system of claim 6 wherein a usable duration of said breakably-actuated scent container is measured at a point from breakable-actuation to a no-scent-release condition coincides with one filter change-out interval and provides an indication of a necessary maintenance task.

8. The scented air filter system of claim 1 wherein said breakably-actuated scent container comprises a tube.

9. The scented air filter system of claim 1 wherein said frame comprises cardboard.

10. The scented air filter system of claim 9 wherein said frame comprises two vertically parallel channels and two horizontally parallel channels.

11. The scented air filter system of claim 10 wherein two of said breakably-actuated scent containers are located within said two vertically parallel channels.

12. The scented air filter system of claim 10 wherein two of said breakably-actuated scent containers are located within said two horizontally parallel channels.

13. The scented air filter system of claim 3 wherein said porous filter media comprises a wicking volume defined by a first and a second outer surface wherein said liquid from said breakably-actuated scent container is moved by capillary action from said wicking volume towards said first and second outer surfaces whereby said liquid is dispersible by said airflow.

14. The scented air filter system of claim 9 wherein said frame comprises four of said breakably-actuated scent containers.

15. The scented air filter system of claim 1 wherein said scented air filter system is usable as a particulate impeder for a cold air suction on a furnace.

16. The scented air filter system of claim 1 wherein said scented air filter system is usable as a particulate impeder for a wall vent.

17. The scented air filter system of claim 1 wherein said scented air filter system is usable as a particulate impeder for a cold air suction on an air conditioner.

18. A scented air filter system comprising:
a frame wherein said frame comprises cardboard;
a porous filter media; and
a breakably-actuated scent container wherein said breakably-actuated scent container comprises a tube;
wherein said frame comprises two vertically parallel channels and two horizontally parallel channels;
wherein two of said breakably-actuated scent containers are located within confines of said two vertically parallel channels and said two horizontally parallel channels;
wherein said frame comprises four of said breakably-actuated scent containers;
wherein said frame is squeezably-deformable such that when said breakably-actuated scent container is squeezed within said frame, said breakably-actuated scent container is breakably-actuated to release a liquid from said tube(s);
wherein said scented air filter system is usable as a particulate impeder for a cold air suction on a furnace, for a wall vent and/or for a cold air suction on an air conditioner;
wherein said breakably-actuated scent container comprises a liquid;
wherein said porous filter media comprises a wicking volume defined by a first and a second outer surface, the wicking volume contained within said first and a second outer surface, wherein said liquid from said breakably-actuated scent container is moved by capillary action from said wicking volume towards said first and second outer surfaces whereby said liquid is dispersible by said airflow;
wherein said liquid within said breakably-actuated scent container comprises an oil or a non-oil fluid, or a granular material;
wherein a usable duration of said breakably-actuated scent container is measured from breakable-actuation to a no-scent-release condition which coincides with one filter change-out interval and provides an indication of a necessary maintenance task;
wherein said frame houses and defines an outer perimeter of said porous filter media;
wherein said breakably-actuated scent container is located within inner confines of said frame; and
wherein said breakably-actuated scent container when breakably-actuated creates a dispersible fragrance moveable by an air flow.

19. The scented air filter system of claim 18 comprising a kit having said air filters of varying sizes for use with furnaces and vents; and a set of user instructions.

20. A method of using a scented air filter system comprising the steps of:
removing a used filter from a filter housing when no scent is being released;
squeezing or deforming sides of a frame of a new filter to fracture at least one breakably-actuated scent container to allow scenting material to emit a scent;
inserting said new filter into said filter housing; and
disposing of said used filter.
